(19) United States
(12) Patent Application Publication
(10) Pub. No.: US 2024/0066317 A1
(43) Pub. Date: Feb. 29, 2024

(54) MULTIFUNCTIONAL NEAR-INFRARED ORTHODONTIC ACCELERATOR FOR INHIBITING DENTAL PLAQUE FORMATION

(71) Applicant: Union Hospital, Tongji Medical College, Huazhong University of Science and Technology, Wuhan (CN)

(72) Inventors: LiLi Chen, Wuhan (CN); JinFeng Peng, Wuhan (CN); QingMing Tang, Wuhan (CN)

(21) Appl. No.: 18/111,779

(22) Filed: Feb. 20, 2023

Related U.S. Application Data
(63) Continuation of application No. PCT/CN2023/071433, filed on Jan. 9, 2023.

(30) Foreign Application Priority Data
Aug. 26, 2022 (CN) 2022110311340

(51) Int. Cl. A61N 5/06

(52) U.S. Cl. CPC A61N 5/0624; A61N 5/0603; A61N 2005/0606; A61N 2005/0645; A61N 2005/0659; A61N 2005/0663

(57) ABSTRACT

A multifunctional near-infrared orthodontic accelerator for inhibiting dental plaque formation is provided, including a bottom brace, an outer periodontal brace, an inner periodontal brace, a tooth groove disposed between the outer periodontal brace and the inner periodontal brace, and micro vibrators disposed on a side close to the tooth groove. The outer periodontal brace and the inner periodontal brace are respectively provided with a holding groove therein, which is communicated with openings facing towards the tooth groove. The holding grooves are provided with tooth cleaning bars, which are connected through universal joints and disposed linearly. The tooth cleaning bars are provided with cavities, which are provided with blue light germicidal lamps fitted to inner walls of the cavities. The outer periodontal brace and the inner periodontal brace are equipped with infrared laser lights, and outer peripheries of the tooth cleaning bars are equipped with bristle strips.

### CROSS-REFERENCE TO RELATED APPLICATION

This application claims priority to Chinese patent application No. 202211031134.0 filed on Aug. 26, 2022 and entitled “Multifunctional Near-Infrared Orthodontic Accelerator for Inhibiting Dental Plaque Formation”, the entire contents of which are incorporated herein by reference.

### TECHNICAL FIELD

The disclosure relates to the field of dental orthodontic machine technology, and in particular to a multifunctional near-infrared orthodontic accelerator for inhibiting dental plaque formation.

### BACKGROUND

Jaw deformity in oral cavity is listed as one of three diseases in the oral cavity by World Health Organization (WHO). The incidence of the jaw deformity is up to about 49%, followed by a year-by-year increase. At present, orthodontic technology in the oral cavity is applied for many clinical treatments. However, orthodontic treatment has a slow effect, which shows that tooth movement speed in the orthodontic treatment is generally 0.8 millimeters per month (mm/month) to 1.2 mm/month, and the comprehensive clinical treatment is generally 18 months to 36 months. Therefore, the slow tooth movement and the long clinical treatment are often difficult for patients to accept, especially for adult patients. In addition, the orthodontic treatment produces pain and discomfort in the teeth within a week after each follow-up clinical visit with force, affects chewing, and brings physical and psychological burdens to the patients, causing resistance to the treatment. At the same time, it is difficult to maintain oral hygiene during the orthodontic treatment, resulting from that the patient wears a bracket (and other orthodontic auxiliary instruments, such as arch wires, planting nails, etc.), and the dental orthodontic appliances are prone to adhesion of food, and there are many grooves and gaps, so that it is difficult for the oral cleaning, and it is easy to accumulate bacteria and soft scale around the bracket to promote the dental plaque formation at the junction between the tooth surfaces and gingivae, and in the gaps of the teeth, thereby to cause gum redness, bleeding, recession, tooth sensitivity, and even alveolar bone resorption and tooth loosening, seriously affecting the orthodontic treatment and oral health. Among the patients, children who are not yet aware of oral hygiene management and adult patients with periodontitis or accompanying systemic diseases such as diabetes are prone to accumulate the dental plaque during the orthodontic treatment, thereby to aggravate periodontal disease and periodontal tissue destruction. As the diet mode changes and the life rhythm is accelerated, the prevalence of periodontitis and diabetes is increased year by year, and the prevalence of diabetes and pre-diabetes exceeds 50%, and more than 90% of adults have periodontal disease of different degrees. Therefore, seeking methods that can safely and efficiently accelerate the tooth movement speed in the orthodontic treatment, reduce the pain after adding force, and effectively inhibit the dental plaque formation is urgent in the orthodontic treatment.

Current research has found that low-intensity and high-frequency vibration accelerates local blood flow, promotes the metabolism of inflammatory molecules, thereby to relieve the pain. Research has shown that blue light with a wavelength of 405 nanometers (nm) to 470 nm illustrates a broad-spectrum antibacterial effect on Grain-negative bacteria and Grain-positive bacteria, which not only can kill antibiotic bacteria and bacterial spores, but also has much less harm to mammalian cells than ultraviolet rays. Compared with traditional antibacterial drugs, the blue light also has the following advantages. Firstly, bacteria in the bacterial biofilm are better killed or reduced. Secondly, no literature reports that the bacteria generate resistance to the blue light. Thirdly, the light beams are confined to the area of bacterial infection and other non-infected parts are not influenced. Fourthly, the illumination radiation intensity is well adjusted and monitored, and the safety of the blue light is higher compared with that of the antibacterial drugs. The mechanism by which blue light kills antibiotic-resistant bacteria may be that the blue light can be absorbed by porphyrin produced by the bacteria, resulting in increased free radicals, thereby causing damage to bacteria. Patent No. ZL202110840674.2 provides an adjustable near-infrared orthodontic accelerator for precision treatment, including: a bottom brace and an outer periodontal brace. The bottom brace and the outer periodontal brace together form a U-shaped brace, the outer periodontal brace has a light-transmitting window in its middle position, and the light-transmitting window is provided with sliding slots on its upper and lower sides of the outer periodontal brace. The sliding slots are slidably provided with a number of laser emitting devices side by side, each the laser emitting devices includes a base and sliding plates disposed on upper and lower ends of the base. The sliding plates are disposed in the sliding slots correspondingly, and the base is provided with infrared laser lamps. The above described accelerator takes advantages of low-intensity near-infrared light to irradiate the teeth to accelerate their movement. While the near-infrared light assists the orthodontic treatment, it can also adjust the laser irradiation area to make the laser action range more accurate. Therefore, accuracy and speed of the orthodontic treatment are guaranteed. Furthermore, an increased temperature of the material is small after the long-term use, so that the material guarantees its safety, and the light source is tightly attached to the gingivae, so that the patients do not need to achieve retention through occlusion. Therefore, the accelerator wears comfortable, its size is small, and it is convenient to wear the accelerator. However, the orthodontic accelerator cannot significantly relieve pain, sterilize, and neither clean the surfaces of the teeth and the orthodontic accelerator. In addition, bacteria and soft scales are easily accumulated around the bracket and at the junction between the tooth surfaces and the gingivae in the orthodontic treatment, resulting in dental plaque formation, affecting oral hygiene and tooth health.

### SUMMARY

The disclosure provides a multifunctional near-infrared orthodontic accelerator for inhibiting dental plaque formation. By adopting one or more technical solutions of the disclosure, it is possible to solve the following problems. Firstly, surfaces of teeth and orthodontic appliances cannot be cleaned; secondly, bacteria and soft scales are easily accumulated around the tooth brackets and at the junction between tooth surfaces and gingivae during the orthodontic process, thereby to cause dental plaque formation, affecting oral health and generating tooth pain in the early orthodontic process.

According to an embodiment of the disclosure, the multifunctional near-infrared orthodontic accelerator for inhibiting dental plaque formation includes a bottom brace, an outer periodontal brace and an inner periodontal brace. The bottom brace, the outer periodontal brace and the inner periodontal brace are configured to form a U-shaped brace together. A tooth groove is disposed between the outer periodontal brace and the inner periodontal brace. Holding grooves are disposed in each of the outer periodontal brace and the inner periodontal brace respectively, openings are disposed on each of the outer periodontal brace and the inner periodontal brace, and the openings are facing towards the tooth groove and are communicated with the holding groove. The holding grooves are provided with a plurality of tooth cleaning bars therein, which are connected through a plurality of universal joints and are disposed linearly. Driving motors are disposed in the outer periodontal brace and the inner periodontal brace, and are connected to the plurality of tooth cleaning bars. The plurality of tooth cleaning bars is provided with cavities therein, the cavities are provided with blue light germicidal lamps therein, and the blue light germicidal lamps fit to inner walls of the cavities. The outer periodontal brace and the inner periodontal brace are further provided with a plurality of first infrared laser lamps therein. Peripheries of the plurality of tooth cleaning bars are provided with bristle strips respectively and the outer periodontal brace is provided with a switch.

### BRIEF DESCRIPTION OF DRAWINGS

In order to more clearly illustrate the technical solutions in the embodiments of the disclosure, the attached drawings that need to be used in the embodiments are briefly described below. Apparently, the attached drawings in the following description are some embodiments of the disclosure, and other drawings may be obtained from them without creative effort to those skilled in the related art.

FIG. 1 illustrates an overall schematic structural diagram of a near-infrared orthodontic accelerator for inhibiting dental plaque formation according to an embodiment of the disclosure.

FIG. 2 illustrates a top cross-sectional view of the near-infrared orthodontic accelerator for inhibiting dental plaque formation shown in FIG. 1.

FIG. 3 illustrates a longitudinal cross-sectional view of the near-infrared orthodontic accelerator for inhibiting dental plaque formation shown in FIG. 1.

FIG. 4 illustrates a transverse partial cross-sectional view of the near-infrared orthodontic accelerator for inhibiting dental plaque formation shown in FIG. 1.

FIG. 5 illustrates a partially enlarged cross-sectional view of a tooth cleaning bar of the near-infrared orthodontic accelerator for inhibiting dental plaque formation shown in FIG. 1.

FIG. 6 illustrates a cross-sectional view of the near-infrared orthodontic accelerator for inhibiting dental plaque formation shown in FIG. 1 while using.

### DESCRIPTION OF REFERENCE NUMERALS

1: bottom brace; 2: outer periodontal brace; 3: inner periodontal brace; 4: tooth groove; 5: holding groove; 6: opening; 7: universal joint; 8: tooth cleaning bar; 9: driving motor; 10: cavity; 11: blue light germicidal lamp; 12: first infrared laser lamp; 13: transparent bristle strip; 14: suction hole; 15: negative pressure aspirator; 16: connecting channel; 17: collection box; 18: second infrared laser lamp; 19: heat conduction layer; 20: switch; 21: micro vibrator; 22: bristle.

### DETAILED DESCRIPTION OF EMBODIMENTS

Technical solutions in embodiments of the disclosure will be clearly and completely described below in conjunction with the attached drawings used in the embodiments of the disclosure, the described embodiments are only some embodiments of the disclosure, and not all embodiments of the disclosure. Based on the described embodiments in the disclosure, all other embodiments obtained by those skilled in the art without creative effort should belong to the scope of protection of the disclosure.

It needs to be noted that all directional indications in the described embodiments of the disclosure are used only to explain relative positional relationship among components and motion of the components in a particular attitude, and if the particular attitude is changed, the directional indications change accordingly.

Furthermore, descriptions such as those involving “first” and “second” in the disclosure are for descriptive purposes only and are not to be construed as indicating or implying their relative importance or implicitly specifying the number of technical features indicated. Thus, the features defined with “first” and “second” may explicitly or implicitly include at least one such feature. In addition, the technical solutions of various described embodiments may be combined with each other, but only on the basis that those skilled in the art can realize. When a combination of technical solutions appears to be contradictory or unrealizable, such combination is regarded not to exist, and is not within the scope of protection of the disclosure.

The disclosure will be described in detail by referring to illustrated embodiments in combination with the attached drawings.

In one aspect of the disclosure, a multifunctional near-infrared orthodontic accelerator for inhibiting dental plaque formation is provided. By adopting one or more technical solutions of the disclosure, it is possible to solve the following problems. Firstly, surfaces of teeth and orthodontic accelerator cannot be cleaned; secondly, bacteria and soft scales are easily accumulated around the bracket and the junction between tooth surfaces and gingivae during the orthodontic treatment, thereby to cause dental plaque formation, affecting oral health and generating tooth pain in the early orthodontic process.

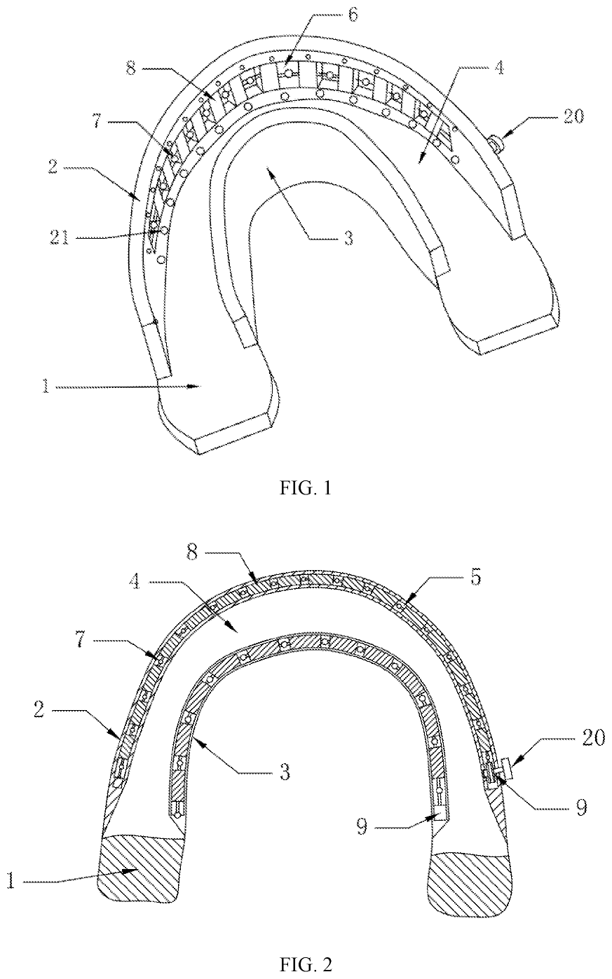

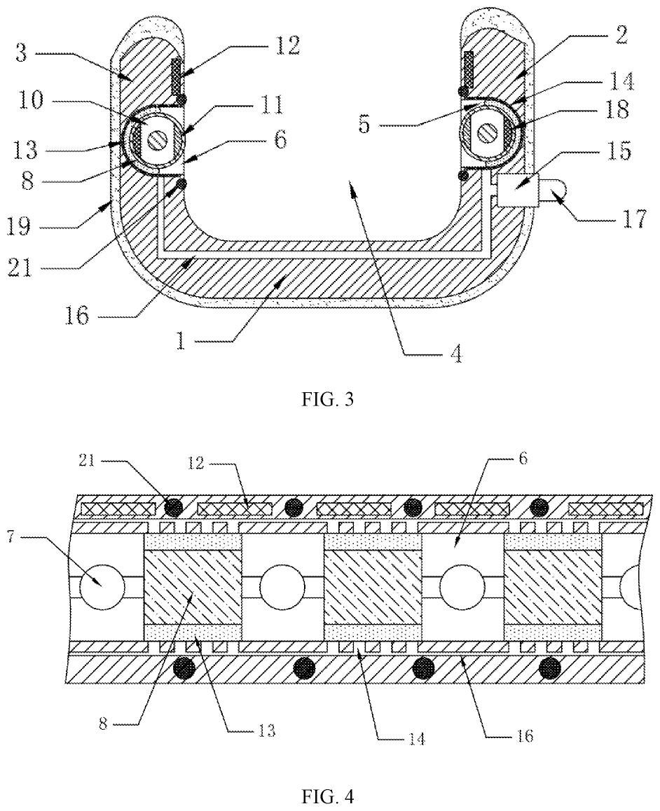

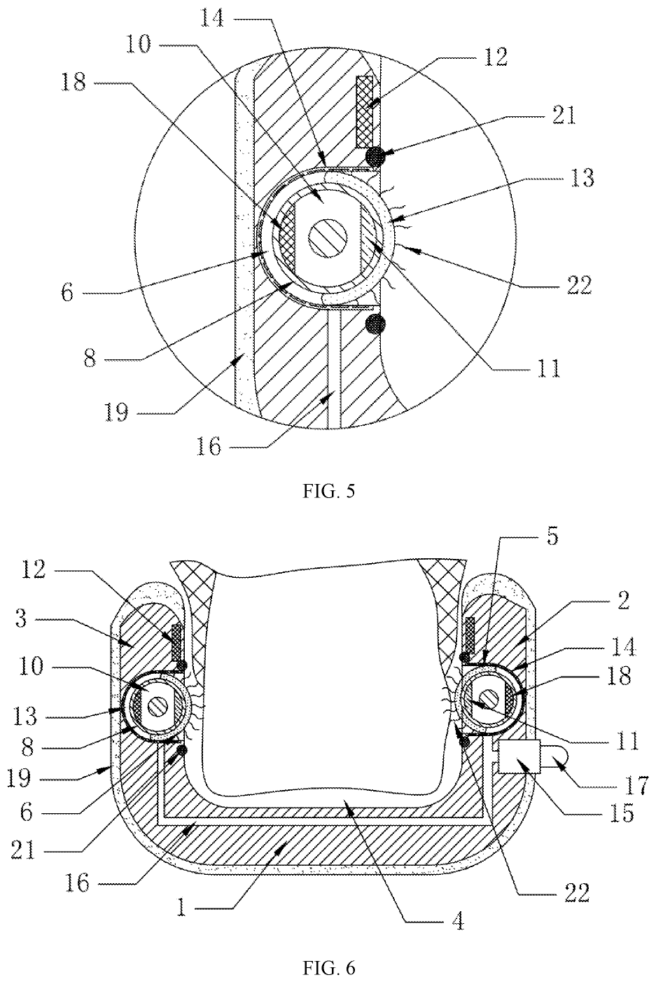

Referring to FIGS. 1-6, the multifunctional near-infrared orthodontic accelerator for inhibiting dental plaque formation includes: a bottom brace 1, an outer periodontal brace 2 and an inner periodontal brace 3, and the bottom brace 1, the outer periodontal brace 2 and the inner periodontal brace 3 together form a U-shaped brace. A tooth groove 4 is disposed between the outer periodontal brace 2 and the inner periodontal brace 3. Each of the outer periodontal brace 2 and the inner periodontal brace 3 is provided with a holding groove 5 therein, openings 6 are disposed on each of the outer periodontal brace 2 and the inner periodontal brace 3, and the openings 6 are facing towards the tooth groove 4 and are communicated with the holding groove 5. Each of the holding grooves 5 is provided with a plurality of tooth cleaning bars 8, which are connected through a plurality of universal joints 7 and are disposed linearly. Driving motors 9 are disposed on the outer periodontal brace 2 and the inner periodontal brace 3 respectively (i.e. two drive motors 9), and are connected to the plurality of tooth cleaning bars 8. Cavities 10 are respectively disposed in the plurality of tooth cleaning bars 8, the cavities 10 are provided with blue light germicidal lamps 11 therein correspondingly, and the blue light germicidal lamps 11 fit to inner walls of the cavities 10. The outer periodontal brace 2 and the inner periodontal brace 3 are further provided with a plurality of first infrared laser lamps 12. Peripheries of the plurality of tooth cleaning bars 8 are provided with transparent bristle strips 13, and a switch 20 is disposed on the outer periodontal brace 2.

In some embodiments, the U-shaped brace formed by the bottom brace 1, the outer periodontal brace 2 and the inner periodontal brace 3 is used to sleeve on the teeth of the patient in the orthodontic treatment. The teeth of the patient are disposed in the tooth groove 4, the tooth cleaning bars 8 are used to rotate in the holding grooves 5 driven by the two driving motors 9. Sides of the transparent bristle strips 13 extend out of the holding grooves 5 through the openings 6, and are fitted to the external tooth surfaces and gingivae of the patient, thereby to clean them. Lifhg rays emitted by the blue light germicidal lamps 11 can pass through the transparent tooth cleaning bars 8 for killing the bacteria accumulated on the teeth. The first infrared laser lamps 12 are used to irradiate gingival roots of the patient with the near infrared radiation to improve the tooth movement speed in the orthodontic treatment. The switch 20 is used to control the working modes of the driving motor 9, the blue light germicidal lamps 11 and the first infrared laser lamps 12. The driving motor 9 can rotate continuously for tooth cleaning under the control of the switch 20, or switch to a mode of rotating at a certain interval with a fixed angle to control the irradiation time of the blue light germicidal lamps 11, thereby to ensure the sterilization effect and avoid damage of normal cells caused by long-term irradiation. In the orthodontic treatment, the bacteria and soft scales accumulated around the bracket or at the junction between the tooth surfaces and the gingivae can be effectively suppressed and cleaned by the blue light germicidal lamps 11 and the tooth cleaning bars 8, so that the oral health is guaranteed in the orthodontic treatment.

In some embodiments of the disclosure, inner walls of the holding grooves 5 are communicated with the plurality of the transparent bristle strips 13, and the inner walls of the holding grooves 5 are provided with a plurality of suction holes 14, which are communicated with the holding grooves 5. The outer periodontal brace 2 is provided with a negative pressure aspirator 15, the suction holes 14 are communicated with the negative pressure aspirator 15 through a connecting channel 16 disposed in the bottom brace 1, the outer periodontal brace 2 and the periodontal brace 3. And the negative pressure aspirator 15 is provided with a collection box 17 thereon. The tartar or bacteria and other substances scraped off from the teeth or gingivae of the patient by the transparent bristle strips 13 are drawn into the collection box 17 through negative pressure suction of the negative pressure aspirator 15, and when the transparent bristle strips 13 rotate to communicate with the suction holes 14 on the inner walls of the holding grooves 5, and the collection box 17 can be removed from the negative pressure aspirator 15 for cleaning to ensure good teeth cleaning effect.

In some embodiments of the disclosure, the transparent bristle strips 13 are made of loose and porous flexible silica gel or sponge, so that the tooth cleaning effect is guaranteed, meanwhile, good air permeability is achieved, and the negative pressure aspirator 15 can suck away the tartar on the transparent bristle strips 13 through the negative pressure suction.

In some embodiments of the disclosure, the cavities 10 are internally provided with second infrared laser lamps 18, which are attached to the inner walls of the cavities 10 and are opposite to the blue light germicidal lamps 11. The driving motors 9 drive the tooth cleaning bars 8 to rotate with a certain angle after the irradiation of the teeth of the patient by the blue light germicidal lamps 11 is finished, and then the positions of the blue light germicidal lamps 11 and the second infrared laser lamps 18 are exchanged, so that the second infrared laser lamps 18 irradiate the tooth surfaces of the patient to cooperate with the first infrared laser lamps 12 to respectively irradiate different positions on the teeth of the patient to enhance the infrared accelerated orthodontic treatment.

In some embodiments of the disclosure, the driving motors 9 are provided with two working modes of a timed working mode and a continuous working mode. When the driving motors 9 are in the timed working mode, a rotating interval is a half circle every 10 minutes. When the driving motors 9 are in the continuous working mode, the tooth cleaning bars 8 rotate continuously and the negative pressure aspirator 15 works to clean the teeth of the patient. When the drive motors 9 are in the timed working mode, the irradiation time of both the blue light and the red light is 10 minutes, alternately sterilizing the tooth surfaces of the patient and accelerating the near infrared. The light emitted by the blue light germicidal lamps 11 is close to the tooth surfaces, the bracket, a junction between the teeth and gingivae, as well as the gaps, gingival sulcus and other places prone to plaque deposition. The light emitted by the first infrared laser lamps 12 is close to the gingival roots of the teeth and the alveolar bone corresponding to the gingivae, and the light emitted by the second infrared laser lamps 18 is close to the tooth surfaces, the combined effect of the two of which accelerates the orthodontic treatment.

In some embodiments of the disclosure, the transparent bristle strips 13 cover half circles of peripheries of the tooth cleaning bars 8, and correspond to positions of the second infrared laser lamps 8, so that light rays of the blue light germicidal lamps 11 are not shielded, thereby to avoid affecting the blue light germicidal lamps 11 working.

In some embodiments of the disclosure, light emitted by the blue light germicidal lamps 11 is blue light with a wavelength of 420 nanometers (nm), which not only kills antibiotic-resistant bacteria and bacterial spores, but also causes little harm to cells and is highly safe. The lights emitted by the first infrared laser lamps 12 and the second infrared laser lamps 18 are near-infrared laser with a wavelength of 810 nm, and a power density of each of the first infrared laser lamps 12 and the second infrared laser lamps 18 is 50 million watt per square centimeters (mW/cm2) to 100 mW/cm2, the strength of which is safe and reliable. Furthermore, the irradiation of the infrared laser lamps does not produce pain.

In some embodiments of the disclosure, outer surfaces of the bottom brace 1, the outer periodontal brace 2 and the inner periodontal brace 3 are provided with heat conduction layers 19, which are plastic and elastic. The tooth cleaning bars 8 are made of transparent plastic, and the heat conduction layers 19 are made of heat conduction silicone with high thermal conductivity, which has a little temperature variation during the use process and prevents discomfort caused by high temperature.

In some embodiments of the disclosure, a plurality of micro vibrators 21 are disposed on sides of the outer periodontal brace 2 and the inner periodontal brace 3 close to the tooth groove 4. Parameters of each the micro vibrator 21 are 25 grains (g), 30 hertz (Hz), providing gentle vibration with light force and high frequency. The micro vibrators 21 are controlled on and off by the switch 20 and are used for the vibration orthodontic treatment, which is achieved by massaging the gingivae through the high-frequency low-intensity vibration to promote local blood circulation, thereby to reduce the pain during the orthodontic treatment.

In some embodiments of the disclosure, the transparent bristle strips 13 are detachably connected to the tooth cleaning bars 8 and surfaces of the transparent bristle strips 13 are provided with a plurality of types of bristles 22. The bristles 22 disposed on the transparent bristle strips 13 can be made of coarse and hard plastic bristles, and can also be made of soft and elongated silica gel bristles. Therefore, it is possible to replace different transparent bristle strips 13 according to different types of the tartar to be cleaned.

In addition, the switch 20 not only can control the working mode of the driving motors 9, but also can control the blue light germicidal lamps 11, the first infrared laser lamps 12, the second infrared laser lamps 18 and the micro vibrators 21 to switch various modes, so that a user can conveniently clean the teeth according to needs, orthodontic acceleration and vibration massage are facilitated, and the use convenience is improved.

The use method of the multifunctional near-infrared orthodontic accelerator for inhibiting dental plaque formation according to the disclosure is as follows. The patient puts the accelerator into the oral cavity, the teeth are placed in the tooth groove 4, plastic and elastic heat conduction layers are disposed on the outer sides of the bottom brace 1, the outer periodontal brace 2 and the inner periodontal brace 3 respectively, so that the accelerator is waterproof and non-conductive. The width of the bottom brace 1 is slightly smaller than the gingival width of the patient, the accelerator can be directly fixed on the teeth under the action of the elasticity of the bottom brace 1 after wearing, which needs no continuous occlusion, and the accelerator is suitable for various tooth arrangement shapes with different deformities. The teeth of the patient are surrounded on the three sides by the bottom brace 1, the outer periodontal brace 2 and the inner periodontal brace 3, and the outer periodontal brace 2 is disposed on the outer side of the patient's teeth. The first infrared laser lamps 12 continuously emit near infrared laser with the wavelength of 810 nm and the intensity of 50 mW/cm² to 100 mW/cm² to irradiate the gingivae corresponding to the tooth roots and the alveolar bone, thereby to promote the tooth movement to realize the orthodontic treatment. The user controls the working mode of the drive motors 9 through the switch 20, and the drive motors 9 can switch between the two working modes of the timed working mode and the continuous working mode. In the timed working mode, the tooth cleaning bars 8 rotate regularly driven by the driving motors 9 for half a circle every 10 minutes. After the blue light germicidal lamps 11 irradiate the tooth surfaces for 10 minutes, the tooth surfaces are cleaned, and then the irradiation of the blue light is stopped, so that the dead bacteria and dirt on the sterilized tooth surfaces are wiped away and sucked into the collection box 17 by the suction holes 14 through the negative pressure suction to keep the tooth surfaces clean. At the same time, the second infrared laser lamps 18 are rotated to align the tooth surfaces for multi-point infrared laser irradiation to accelerate the orthodontic treatment and to ensure the accuracy and speed of the orthodontic treatment. In the continuous working mode, the driving motors 9 drive the tooth cleaning bars 8 to continuously rotate, and the negative pressure aspirator 15 continuously works to continuously clean the teeth of the patient, thereby to inhibit the dental plaque formation. When the patient has soreness on the teeth after the orthodontic force, the micro vibrators 21 can be controlled to work through the switch 20, so that the teeth are vibrated and massaged, effectively relieving the soreness.

Compared with the related art, the disclosure has the following advantages. Firstly, the disclosure includes the U-shaped brace consisting of the bottom brace, the outer periodontal brace and the inner periodontal brace; the holding grooves are disposed in the outer periodontal brace and the inner periodontal brace respectively; the openings are disposed on the outer periodontal brace and the inner periodontal brace respectively, are facing towards the tooth groove and are communicated with the holding grooves; the plurality of tooth cleaning bars are disposed in the holding grooves, are connected through a plurality of universal joints and are linearly arranged; the driving motors are disposed in the outer periodontal brace and the inner periodontal brace respectively, and are connected to the tooth cleaning bars; the cavities are disposed in the plurality of tooth cleaning bars and are provided with blue light germicidal lamps therein; the plurality of first near infrared laser lamps are further disposed in the outer periodontal brace and the inner periodontal brace; the peripheries of the plurality of tooth cleaning bars are provided with the flexible transparent bristle strips, the above descriptions of which realize killing the bacteria on the teeth by the blue light germicidal lamps, cleaning the dead bacteria through the interval rotation of the tooth cleaning bars, while using low-intensity near-infrared light to irradiate the teeth to assist the orthodontic treatment, thereby effectively preventing the bacteria and soft scales from forming the dental plaque at the junction between the tooth surfaces and gingivae and in the crevices during the orthodontic treatment, maintaining the oral health and improving the orthodontic treatment. Secondly, by setting the micro vibrators, it can effectively relieve the soreness of teeth during the orthodontic treatment. Thirdly, the disclosure has various functions, different working modes are provided to select according to needs, and the corresponding function can be realized by simply selecting the corresponding mode. The pain can be alleviated by the vibration in the early stage after the orthodontic force, and the sterilization can be realized throughout the orthodontic treatment. Furthermore, the accelerator is small, easy to adjust, simple to operate, non-invasive and painless, which helps inhibit the dental plaque formation during the orthodontic treatment, greatly reduces the orthodontic risk in patients with periodontitis and diabetes, and provides practical solutions for clinical improvement of orthodontic efficacy, shortening the orthodontic treatment course and reducing orthodontic complications.

In the description of the disclosure, it needs to be understood that the terms “center”, “longitudinal”, “transverse”, “length”, “width”, “thickness”, “up”, “down”, “front”, “back”, “left”, “right”, “vertical”, “horizontal”, “top”, “bottom”, “inside”, “outside, “clockwise”, “counterclockwise”, and the like indicate orientation or positional relationships, which are based on the attached drawings. In addition, the terms are intended only to facilitate the description of the disclosure and simplify the description, and are not intended to indicate or imply that the devices or components referred to must have a particular orientation, be constructed and operated in a particular orientation. Therefore, the terms cannot be understood as limiting the disclosure.

In the description of the disclosure, unless otherwise expressly specified and limited, the first feature “on” or “under” the second feature may include a direct contact between the first feature and the second feature, or may include that the first feature and the second feature are not in contact directly, but through another features between them. Also, the first feature being “on”, “upper” and “above” the second feature includes the first feature being directly above and diagonally above the second feature, or simply indicating that the first feature is horizontally higher above the second feature. The first feature being “under”, “lower” and “below” the second feature includes the first feature being directly below and diagonally below the second feature, or simply indicating that the first feature is less than the horizontal height of the second feature.

In the description of the specification, reference to the terms “an embodiment”, “some embodiments”, “example”, “the illustrated embodiment”, or “some examples”, the above descriptions mean that the specific features, structures, materials, or characteristics described in connection with the embodiment or example are included in at least one embodiment or example of the disclosure. In the specification, the schematic representation of the above terms need not be directed to the same embodiment or example. Moreover, the specific features, structures, materials, or characteristics described may be combined in a suitable manner in any one or more embodiments or examples. In addition, those skilled in the related art may join and combine the different embodiments or examples described in the specification.

Although the illustrated embodiments of the disclosure have been described, those skilled in the related art can make additional changes and modifications to these embodiments based on basic concepts. Therefore, the appended claims are intended to be construed to include the illustrated embodiments and all changes and modifications that fall within the scope of the present disclosure.

It is clear that those skilled in the related art can make various modifications and variations to the disclosure without departing from the spirit and scope of the disclosure. Therefore, if the various modifications and variations of the disclosure fall within the scope of the claims of the disclosure and their technical equivalents, the disclosure is also intended to encompass such modifications and variations.

What is claimed is:

1. A near-infrared orthodontic accelerator for inhibiting dental plaque formation, comprising: a bottom brace, an outer periodontal brace and an inner periodontal brace;
wherein the bottom brace, the outer periodontal brace and the inner periodontal brace are configured to form a U-shaped brace together;
wherein a tooth groove is disposed between the outer periodontal brace and the inner periodontal brace;
wherein holding grooves are disposed in each of the outer periodontal brace and the inner periodontal brace respectively;
wherein openings are disposed on each of the outer periodontal brace and the inner periodontal brace, the openings are facing towards the tooth groove and are communicated with a corresponding one of the holding groove;
wherein each of the holding grooves is provided with a plurality of tooth cleaning bars therein, which are connected through a plurality of universal joints and are disposed linearly;
wherein a driving motor is disposed in each of the outer periodontal brace and the inner periodontal brace, and is connected to the plurality of tooth cleaning bars;
wherein each of the plurality of tooth cleaning bars is provided with a cavity therein, the cavity is provided with a blue light germicidal lamp therein, and the blue light germicidal lamp fits to an inner wall of the cavity;
wherein each of the outer periodontal brace and the inner periodontal brace is further provided with a plurality of first infrared laser lamps therein;
wherein peripheries of the plurality of tooth cleaning bars are provided with bristle strips, respectively; and
wherein the outer periodontal brace is provided with a switch.

2. The near-infrared orthodontic accelerator for inhibiting dental plaque formation according to claim 1, wherein the bristle strips are in contact with an inner wall of the holding groove and the inner wall of the holding groove is provided with a plurality of suction holes communicated with the holding groove;
wherein the outer periodontal brace is provided with a negative pressure aspirator thereon;
wherein the suction holes are communicated with the negative pressure aspirator through a connecting channel disposed in the bottom brace, the outer periodontal brace and the inner periodontal brace; and
wherein the negative pressure aspirator is provided with a collection box thereon.

3. The near-infrared orthodontic accelerator for inhibiting dental plaque formation according to claim 2, wherein the bristle strips are made of one of loose and porous flexible silica gel and sponge.

4. The near-infrared orthodontic accelerator for inhibiting dental plaque formation according to claim 1, wherein the cavity is provided with a second infrared laser lamp therein, which is attached to the inner wall of the cavity and opposites to the blue light germicidal lamp.

5. The near-infrared orthodontic accelerator for inhibiting dental plaque formation according to claim 1, wherein the driving motor is provided with two working modes of a timed working mode and a continuous working mode, and when the driving motor is in the timed working mode, a rotating interval of the driving motor is a half circle every 10 minutes.

6. The near-infrared orthodontic accelerator for inhibiting dental plaque formation according to claim 4, wherein each of the bristle strips covers a half circle of the periphery of a corresponding one of the tooth cleaning bars, and corresponds to a position of the second infrared laser lamp, so that a light ray emitted by the blue light germicidal lamp is not shielded.

7. The near-infrared orthodontic accelerator for inhibiting dental plaque formation according to claim 4, wherein a light ray emitted by the blue light germicidal lamp is blue light with a wavelength of 420 nanometers (nm), light rays emitted by the first infrared laser lamp and the second infrared laser lamp are near-infrared laser with a wavelength of 810 nm, and a power density of each of the first infrared laser lamp and the second infrared laser lamp is 50 million watt per square centimeters (mW/cm²) to 100 mW/cm².

8. The near-infrared orthodontic accelerator for inhibiting dental plaque formation according to claim 1, wherein outer surfaces of the bottom brace, the outer periodontal brace and the inner periodontal brace are provided with heat conduction layers, the heat conduction layers are plastic and elastic, and the tooth cleaning bars are made of transparent plastic.

9. The near-infrared orthodontic accelerator for inhibiting dental plaque formation according to claim 1, wherein a plurality of micro vibrators are disposed on sides of the outer periodontal brace and the inner periodontal brace close to the tooth groove.

10. The near-infrared orthodontic accelerator for inhibiting dental plaque formation according to claim 1, wherein the bristle strips are detachably connected to the tooth cleaning bars and surfaces of the bristle strips are provided with a plurality of types of bristles.
